(19) United States
(12) Patent Application Publication
(10) Pub. No.: US 2026/0198966 A1
(43) Pub. Date: Jul. 16, 2026

(54) SYSTEMS AND METHODS FOR HAIR TRANSPLANTATION BY SINGLE DEVICE WITH COUPLER

(71) Applicant: The General Hospital Corporation, Boston, MA (US)

(72) Inventors: Sandeep Korupolu, Boston, MA (US); William Farinelli, Boston, MA (US); Walfre Franco, Boston, MA (US); Lynn Drake, Boston, MA (US); Richard Rox Anderson, Boston, MA (US)

(21) Appl. No.: 19/127,802

(22) PCT Filed: Nov. 8, 2023
(86) PCT No.: PCT/US2023/079066

§ 371 Date: May 7, 2025

Related U.S. Application Data
(60) Provisional application No. 63/382,877, filed on Nov. 8, 2022.

(51) Int. Cl. A61B 17/34; A61B 17/00

(52) U.S. Cl. CPC A61B 17/3468; A61B 2017/0023; A61B 2017/00752; A61B 2017/00969

(57) ABSTRACT

Hair transplant systems and methods include or use an extracting unit including: a first housing extending from a proximal end portion to a distal end portion, wherein the distal end portion includes an end notch defining a seat portion and a step portion, an extracting needle, and a first coupler; an implanting unit including: a second housing, an implanting needle, and a second coupler configured to be received by the first coupler; and a resilient element disposed between the extracting unit and the implanting unit to provide a biasing force to cause the hair transplant system to move between an extraction state and an implantation state. In the extraction state, the implanting unit is positioned within the seat portion of the distal end portion, and in the implantation state, the implanting unit is positioned on the step portion of the distal end portion.

### CROSS-REFERENCE TO RELATED APPLICATIONS

This application claims the benefit of and priority to U.S. Provisional Patent Application No. 63/382,877, filed on Nov. 8, 2022, entitled “Singular device with coupler for Hair transplantation,” the entire contents of which are herein incorporated by reference for all purposes.

### TECHNICAL FIELD

This disclosure relates to the field of hair transplantation. More specifically, this disclosure relates to systems and methods for hair transplantation using a single device for both hair extraction and hair implantation.

### BACKGROUND

Hair loss is a physical and psychological issue that affects 80 million people in the United States alone. As a result, the commercial market for addressing hair loss is a multi-billion dollar industry, from drug therapies to hair transplantation.

Hair transplantation is a procedure that involves implanting multiple hair follicles or follicular units, from a donor site of a donor, into a recipient site of a patient. Generally, this procedure is done by first identifying a hair follicle in the donor site, coring around the hair follicle, and removing the hair follicle from the donor site. Then, a small opening is created in the recipient site. After the small opening has been created the hair follicle is implanted within the opening, and the opening is allowed to heal around the implanted hair follicle.

More specifically, there are two types of hair transplantation procedures: Follicular Unit Transplantation (“FUT”), and Follicular Unit Extraction (“FUE”). In FUT, a long strip of skin is cut from the donor site (e.g., usually back of the head) and from the cut skin, the hair follicles are separated. For implanting, small holes are made at the recipient site and the separated hair follicles are inserted into them one at a time. This procedure requires highly skilled surgeons and technicians and leaves a long scar where the skin was cut. In FUE, each hair follicle is identified, and small holes are made using coring punches around the hair follicle and then removing the hair follicle from the donor site one at a time. For implanting, small holes are made at the recipient site and each hair follicle is inserted into the holes. The holes are then allowed to heal around the implanted hair follicle.

In comparative examples, this procedure is performed using differing tools for extraction of the hair follicle, creation of the small opening, and implantation of the hair follicle. Further, the procedure is typically done by extracting a single follicle at a time, temporarily placing each single follicle at a holding location (e.g., a petri dish) one by one, and then implanting a single hair follicle at a time. A single hair transplant session may implant anywhere from 1,500 to 3,000 hair follicles. With each hair follicle taking twenty seconds or longer to transplant, each session is very labor intensive and can last as long as eight to twelve hours. As such, comparative processes for hair transplantation is tedious, time-consuming, and costly. Therefore, it would be advantageous to have systems and methods to reduce the hair transplantation surgery (FUE) time and increase hair transplantation efficiency.

### SUMMARY

The present disclosure overcomes these and other drawbacks by providing systems and methods for hair transplantation using a single device which includes both extraction and implantation features. In some examples, the systems and methods described herein eliminate intermediate stages in the hair transplantation process (e.g., manipulating hair follicles such as by placing them in the holding location prior to implant). Accordingly, the present disclosure provides for systems and methods of hair transplantation which may proceed much more quickly and efficiently than comparative methods. The systems and methods of the present disclosure are provided for improved hair transplant procedures that increase extraction speed, opening speed, and implantation speed, thereby increasing efficacy and reducing cost. Moreover, by reducing the time between extraction and implantation, the systems and methods described herein reduce stress on the hair follicles, which may lead to better transplantation outcomes.

According to one aspect of the present disclosure, a hair transplant system is provided. The hair transplant system comprises an extracting unit including: a first housing extending from a first proximal end portion to a first distal end portion, wherein the first distal end portion includes an end notch defining a seat portion and a step portion, an extracting needle located at the first distal end portion, and a first coupler; an implanting unit including: a second housing extending from a second proximal end portion to a second distal end portion, an implanting needle located at the second distal end portion, and a second coupler configured to be received by the first coupler; and a resilient element disposed between the extracting unit and the implanting unit, the resilient element configured to provide a biasing force to cause the hair transplant system to move between an extraction state and an implantation state, wherein in the extraction state, the second proximal end portion is positioned within the seat portion of the first proximal end portion, and in the implantation state, the second proximal end portion is positioned on the step portion of the first proximal end portion.

According to another aspect of the present disclosure, a hair transplant method is provided. The method comprises providing a hair transplant device at donor site, wherein the hair transplant system comprises an extracting unit including an extracting needle, an implanting unit including an implanting needle, and a resilient element disposed between the extracting unit and the implanting unit and configured to cause the hair transplant device to move between an extraction state and an implantation state, wherein the hair transplant device is provided in the extraction state; piercing a target hair follicle at the donor site with the extracting needle; extracting the target hair follicle into the hair transplant device; and switching the hair transplant device from the extraction state to the implantation state; piercing a recipient site with the implantation needle; and implanting the target hair follicle at the recipient site.

According to another aspect of the present disclosure a method of manufacturing a hair transplant system is provided. The method comprises providing an extracting unit, wherein the extracting unit includes: a first housing extending from a first proximal end portion to a first distal end portion, wherein the first distal end portion includes an end notch defining a seat portion and a step portion, an extracting needle located at the first distal end portion, and a first coupler; providing an implanting unit, wherein the implanting unit includes: a second housing extending from a second proximal end portion to a second distal end portion, an implanting needle located at the second distal end portion, and a second coupler; positioning a resilient element over the first housing; and positioning the implanting unit over the first housing such that the second coupler is received by the first coupler and such that the resilient element is disposed between the extracting unit and the implanting unit, thereby to provide a biasing force to cause the hair transplant system to move between an extraction state and an implantation state, in which: in the extraction state, the second proximal end portion is positioned within the seat portion of the first proximal end portion, and in the implantation state, the second proximal end portion is positioned on the step portion of the first proximal end portion.

According to another aspect of the present disclosure, a hair transplant method is provided. The hair transplant method comprises extracting a target hair follicle from a donor site into a hair transplant device with an extracting needle of a hair transplant device, positioning an implanting needle of the hair transplant device over the extracting needle without manipulating the target hair follicle, and implanting the target hair follicle at a recipient site with the implanting needle.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an example in more detail than is necessary for a fundamental understanding of the teachings of the disclosures. In the drawings:

FIG. 1A is a perspective view of an example of a hair transplant device including an implanting unit and extracting unit in accordance with various aspects of the present disclosure.

FIG. 1B is an exploded view of the hair transplant device of FIG. 1A.

FIG. 2A are views of an example of the extracting unit of FIG. 1A.

FIG. 2B is a view of an example of the extracting unit of FIG. 1A.

FIG. 3A is a view of an example of the implanting unit of FIG. 1A.

FIG. 3B is a view of an example of the implanting unit of FIG. 1A.

FIG. 4 is a perspective view of another configuration of the hair transplant device of FIG. 1A.

FIG. 5 is a flowchart describing an example method of operation, according to various aspects of the present disclosure.

FIG. 6 is an illustration of the example method of operation of FIG. 5.

FIG. 7 is an illustration of the example method of operation of FIG. 5.

FIG. 8 is an illustration of the example method of operation of FIG. 5.

FIG. 9 is an illustration of the example method of operation of FIG. 5.

FIG. 10 is an illustration of the example method of operation of FIG. 5.

FIG. 11 is a schematic of an example of an automated hair transplant system in accordance with various aspects of the present disclosure.

FIG. 12 is a flowchart describing an example method of manufacturing and/or assembly, according to various aspects of the present disclosure.

### DETAILED DESCRIPTION

In the following detailed description, reference is made to the accompanying drawings in which specific examples are shown by way of illustration. These examples are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

Unless otherwise indicated, the various features illustrated in the drawings may not be drawn to scale. The illustrations presented herein are not necessarily intended to be actual views of any particular method, device, or system, but are merely idealized representations that are employed to describe various embodiments of the disclosure. Accordingly, the dimensions of the various features as illustrated may be arbitrarily expanded or reduced for clarity. In addition, some of the drawings may be simplified for clarity. Thus, the drawings may not depict all of the components of a given apparatus (e.g., device) or method. In addition, like reference numerals may be used to denote like features throughout the specification and figures.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “only one of,” or “exactly one of.” For example, a list of “only one of A, B, or C” indicates options of: A, but not B and C; B, but not A and C; and C, but not A and B. In contrast, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more A, one or more B, and one or more C. Similarly, a list preceded by “a plurality of” (and variations thereon) and including “or” to separate listed elements indicates options of one or more of each of multiple of the listed elements. For example, the phrases “a plurality of A, B, or C” and “two or more of A, B, or C” indicate options of: one or more A and one or more B; one or more B and one or more C; one or more A and one or more C; and one or more A, one or more B, and one or more C.

As will be described herein, the present disclosure provides systems and methods for transplanting hair follicles from a donor site to a recipient site using a hair transplant system and method. The hair transplant device described herein includes an extracting unit configured to extract at least one hair follicle from a donor site and an implanting unit removably coupled to the extracting unit, the implanting unit including configured to form an opening into a recipient site.

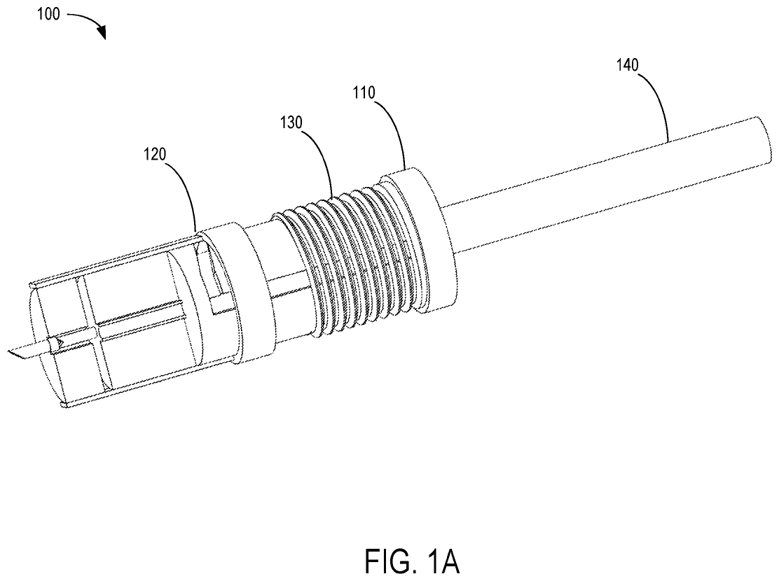

Referring now to the drawings wherein like reference numerals correspond to similar elements throughout the several views and, more specifically, referring to FIGS. 1A and 1B, a hair transplant device 100 (an example of a “hair transplant system” in accordance with the present disclosure) for extracting hair follicles from a donor site of a donor and implanting them into a recipient site of a patient is illustrated. These hair follicles, or follicular units, can contain a single hair or multiple hairs grouped together. The donor and the patient may be the same or different persons. In some examples, the donor site is a portion of the scalp located on the rear of a patient's head, and the recipient site is a portion of the scalp located on the top and/or sides of the same patient's head.

As can be seen in the illustration of FIG. 1A, the hair transplant device 100 includes an extracting unit 110, an implanting unit 120 coupled to the extracting unit 110, a first resilient element 130, a pin 140, and a second resilient element 150. The extracting unit 110 is configured to form an opening into a donor site, thereby to extract a hair follicle, and the implanting unit 120 is configured to form an opening into a recipient site, thereby to implant the hair follicle. The extracting unit 110 and the implanting unit 120 cooperate to form a single device capable of performing both extraction and implantation. FIG. 1A illustrates the hair transplant device 100 in an assembled view, and FIG. 1B illustrates the hair transplant device 100 in an exploded view.

As can be seen in the exploded view of FIG. 1B, the extracting unit 110 includes a first housing 112, which extends from a proximal end portion to a distal end portion. As used herein, the terms “proximal” and “distal” refer to the position along a longitudinal axis of the hair transplant device 100, from the perspective of an operator of the hair transplant device. Thus, in use, the proximal end portion is located nearer to the operator, and the distal end portion is located nearer to the patient. The extracting unit 110 includes an end notch 114 at the distal end portion thereof. An extracting needle 116 is located at the distal end portion. The extracting unit 110 further includes a coupler 118.

In some instances, the first housing 112 may taper toward the distal end portion. In other instances, the first housing 112 may alternatively have a distal end portion that is round, flat, or any other suitable shape.

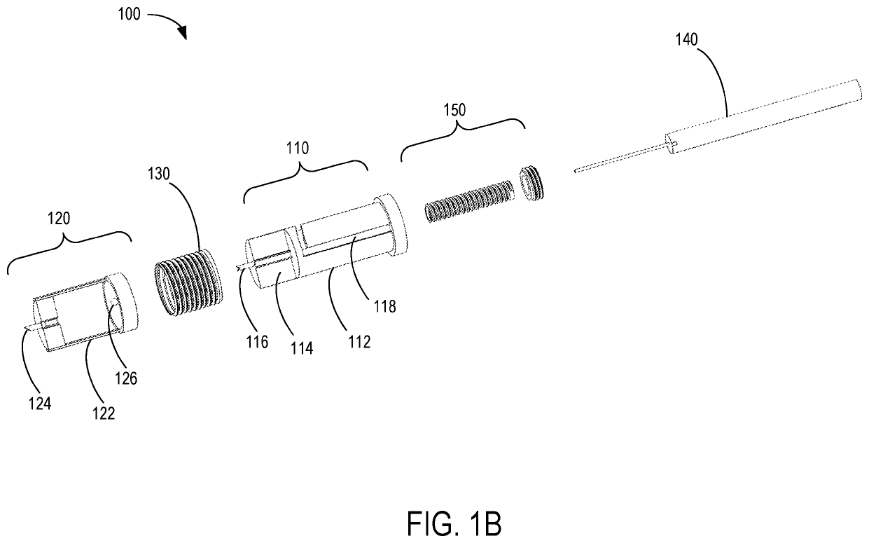

The extracting unit 110 is illustrated in more detail in FIGS. 2A and 2B, in which FIG. 2A shows a top view of the extracting unit 110 and FIG. 2B shows a front view of the extracting unit 110. The extracting unit 110 has a distal end 202 and proximal end 204. The proximal end 204, as illustrated, includes a flanged portion on which the first resilient element 130 may sit. The extracting unit 110 includes a cavity 206 extending from the distal end 202 to the proximal end 204, which is dimensioned to receive a pin of the pin 140 therein. The end notch 114 defines a seat portion 208 and a step portion 210. The extracting needle 116 is located on the step portion 210.

The extracting needle 116 may be a coring needle, formed as a hollow needle with a coring needle lumen extending therethrough. Further, the coring needle may include a first distal cutting end. The distal cutting end is configured to cut into the donor site to form a core in order to extract a hair follicle within the core. Each distal cutting end may include a pair of angled surfaces that angle toward each other, intersecting at the distal end of the coring needle. Accordingly, the pair of angled surfaces form a pair of cutting edges disposed on opposite sides of the coring needle lumen. The pair of cutting edges are effectively aligned across the coring needle, such that they both extend radially from an inner surface of the coring needle to an outer surface of the coring needle. As such, the coring needle can be configured to cut into tissue by driving the coring needle into the tissue, without needing to rotate the coring needle.

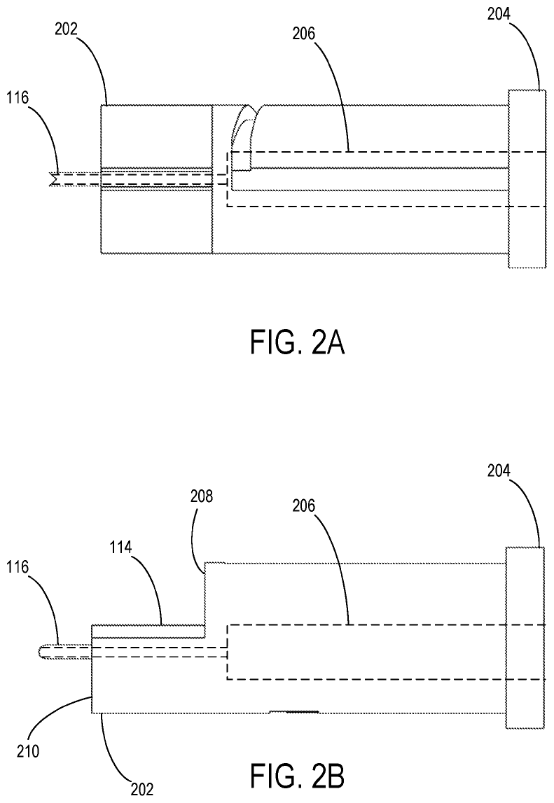

The implanting unit 120 is illustrated in more detail in FIGS. 3A and 3B, in which FIG. 3A shows a top view of the implanting unit 120 and FIG. 3B shows a front view of the implanting unit 120. The implanting unit has a distal end 302 and a proximal end 304. The proximal end 304 is comparatively wide, and thus the implanting unit 120 may sit on the resilient element 130. The implanting needle 124 is located on the face of the implanting unit 120 at the distal end 302.

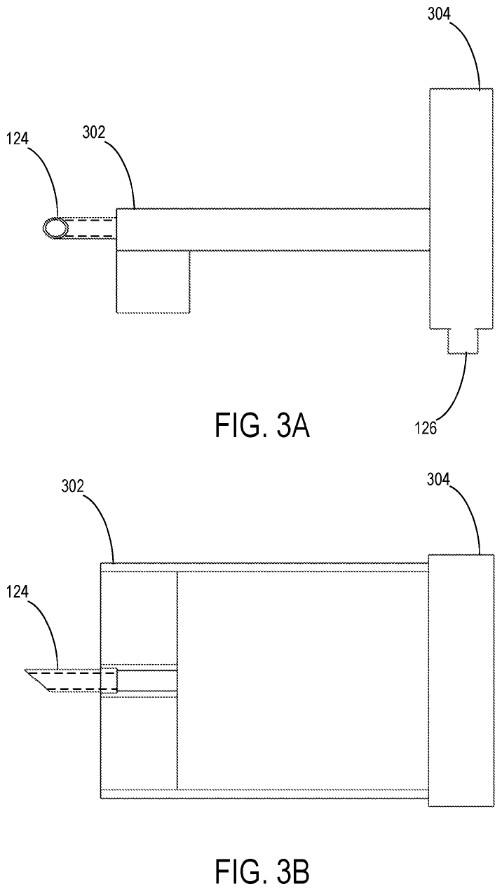

The hair transplant device 100 may be placed in either an extraction state or an implantation state based on the relative position of the extracting unit 110 and the implanting unit 120. The extraction state is illustrated in FIG. 4, and the implantation state is illustrated in FIG. 1A. The first resilient element 130 lies between the extracting unit 110 and the implanting unit 120, and provides a biasing force to cause the hair transplant device 100 to move between the two states. In use, the biasing force causes the implanting unit 120 to slide on the extracting unit 110 with the help of the first coupler 118 and the second coupler 126. The first coupler 118 is illustrated as a groove, and the second coupler 126 is illustrated as a guide protrusion configured to be received in the groove. When the hair transplant device 100 is in the extraction state, the implanting unit 120 is seated on the seat portion 208 and thus located within the end notch 114, and the implanting needle 124 is not exposed, as shown in FIG. 4. When the hair transplant device 100 is in the implantation state, the implanting unit 120 is seated on the step portion 210, and the implanting needle 124 is placed over the extracting needle 116.

The implanting needle 124 may be a splitting needle, formed as a hollow needle with a splitting needle lumen extending therethrough. Further, the splitting needle includes a second distal cutting end that is angled relative to a central axis of the splitting needle. The distal cutting end of the splitting needle is configured to form an opening in a recipient site in order to implant the extracted core. The second distal cutting end has a specific geometry (e.g., the angle of the distal cutting edge) that can prevent tissue from entering the splitting needle while the splitting needle cuts into skin by piercing the skin and gradually pushing the tissue apart, similar to the function of a hypodermic needle.

In the illustrated example, in which the extracting needle 116 is a coring needle and the implanting needle 124 is a splitting needle, the inner diameter of the coring needle and the inner diameter of the splitting needle are substantially the same. That is, the coring needle defines a first diameter that is equal to a second diameter defined by the splitting needle. In other examples, however, the diameters of the coring and splitting needles may be different from one another.

The pin 140 includes a distal end and a proximal end. The pin 140 may be referred to as a “pin” in some implementations. The distal end of the pin 140 is covered with a mesh (not shown), and the proximal end of the pin 140 may have a port to allow a connection to a vacuum source. The mesh can be configured to prevent a core extracted from the donor site from being pulled into a central lumen of the pin 140. For example, if suction is applied to the central lumen (e.g., via the port using the vacuum source), the mesh can provide a surface that allows suction therethrough, but does not allow the passage of objects such as the extracted core. The mesh can define a porous surface including a plurality of holes. The proximal end of the pin 140 is inserted into the cavity 206 of the extracting unit 110.

The second resilient element 150 lies over the pin 140 and within the cavity 206 of the extracting unit 110 and exerts a biasing force. If a user pushes the pin 140, for example to effect implantation of a core as will be described in more detail below, the second resilient element 150 causes the pin 140 to return to its original position. A pin stopper is also illustrated in the exploded view of FIG. 1B, which operates to prevent the pin 140 from falling out of the casing 110 (e.g., due to the biasing force). As illustrated in FIGS. 1A and 1B, the first resilient element 130 and the second resilient element 150 are springs. However, in other implementations the first resilient element 130 and/or the second resilient element 150 may be another device that exerts a biasing force.

Various components of the hair transplant device 100 may be made of metal, plastic, ceramic, or any other suitable materials. For example, the extracting needle 116, the implanting needle 124, and the pin 140 may be made from medical grade or surgical stainless steel. This allows for easy cleaning of the hair transplant device 100. The extracting needle 116 and implanting needle 124 may be equal or different sizes. In some examples, the hair transplant device may include a plurality of extracting needles 116. In some examples, the hair transplant device 100 may include a plurality of implanting needles 124. Any number of extracting needles 116 and/or implanting needles 124 may be provided, limited only by the size of the donor site, the size of the recipient site, and/or the size of the hair transplant device 100. In examples, the number of extracting needles 116 is equal to the number of implanting needles. The plurality of extracting needles 116 and/or implanting needles 124 may be arranged in-line or in a circular or other two-dimensional arrangement. A plurality of in-line needles may be spaced so as to extract hairs that are immediately adjacent (e.g., approximately 1 mm apart), evenly spaced, or may be spaced so as to allow the user to extract hairs that are not immediately adjacent. This can be beneficial because the hairs will be removed at a lower concentration from a greater area, so the patient is not left with a bald spot. The extracting needle 116 and/or implanting needle 124 may also be interchangeable or disposable and replaced after use. In one example the extracting needle 116 and implanting needle 124 are 14 gauge needles. The extracting needle 116 and implanting needle 124 may be exchanged for needles of different sizes. The implanting needle 124 density can be varied. Thus, the hair tissues can be extracted at one density and implanted at a different density. As such the hair transplant device 100 can have a plurality of extracting needles 116 and/or implanting needles 124 of any suitable shape or size, with evenly or unevenly spaced hair transplant devices, as desired by the doctor or other hair transplant procedure personnel.

Now that the general structure of the hair transplant device 100 has been described above, exemplary methods of use will be described below. It should be noted that the methods of use described below are given as examples and are not meant to be limiting in any way.

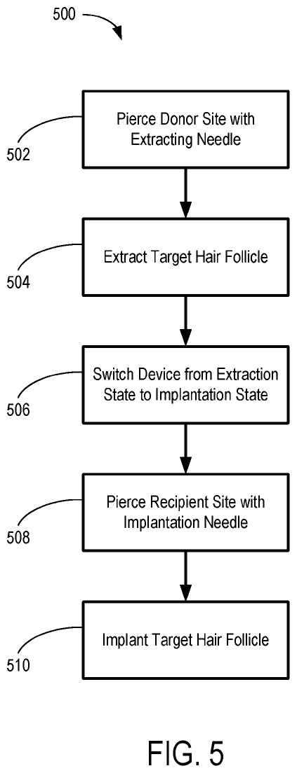

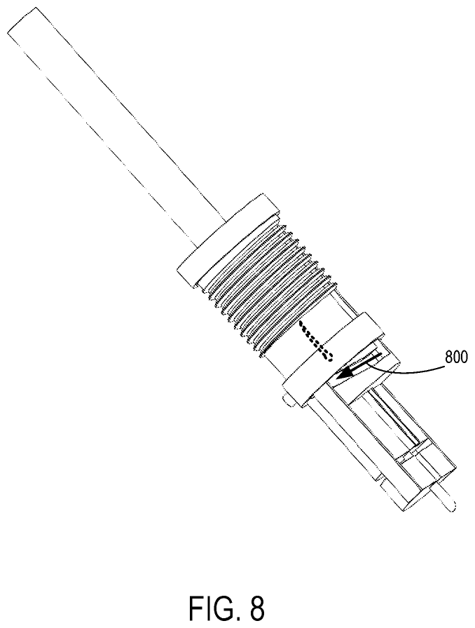

The hair transplant device 100 can be used to perform multiple different procedures to complete a hair transplant operation on a patient. For example, the hair transplant device 100 is designed to perform an extraction procedure (shown in FIGS. 6-8), and an implantation procedure (shown in FIG. 9). Although any one of these two procedures can be performed individually by the hair transplant device 100, the hair transplant device 100 allows for the two procedures to be done sequentially and repetitively. That is, the hair transplant device 100 can first be used to extract a hair follicle from a donor site of a donor during an extraction procedure. Then, the hair transplant device 100 can be used to implant the hair follicle from the donor site into the recipient site. Finally, once the hair follicle has been implanted into the recipient site, the hair transplant device 100 can be used to repeat this process again and again to complete the hair transplant operation. This process may be repeated, for example, tens, hundreds, or even thousands of times.

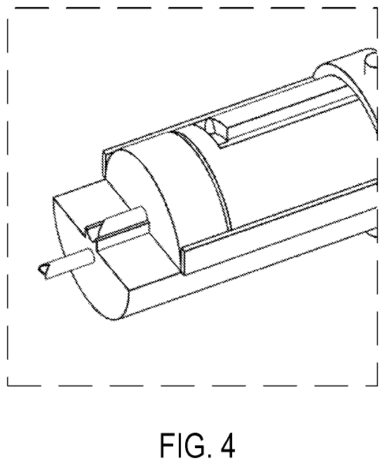

FIG. 5 depicts a method of operation 500 of a hair transplant device. Solely for purposes of explanation, the method 500 will be described as being performed by the hair transplant device 100 of FIGS. 1A-4. Generally, the method 500 may be described as a method of extracting a target hair follicle from a donor site into a hair transplant device with an extracting needle of a hair transplant device, positioning an implanting needle of the hair transplant device over the extracting needle without manipulating the target hair follicle, and implanting the target hair follicle at a recipient site with the implanting needle.

The method 500 begins with providing the hair transplant device 100, which may comprise an extracting unit (e.g., 110 of FIG. 1A) including an extracting needle, an implanting unit (e.g., 120 of FIG. 1A) including an implanting needle, and a resilient element (e.g., 130 of FIG. 1A) disposed between the extracting unit and the implanting unit and configured to cause the hair transplant device to move between an extraction state and an implantation state. The operation of the hair transplant device 100 is performed with the hair transplant device 100 in the home position or extraction position (i.e., an extraction state) This may be the position illustrated in FIG. 4. Operation 502 includes piercing the hair transplant device 100 on the target hair follicle 610 at the donor site 600. This is illustrated in FIG. 6.

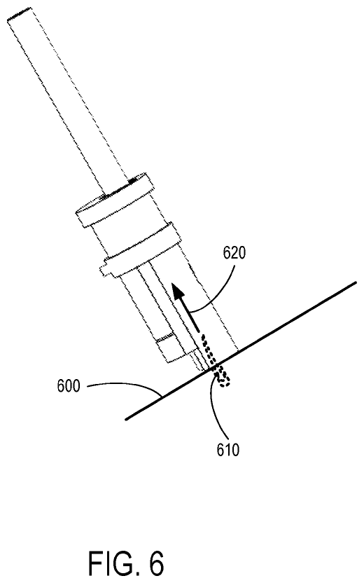

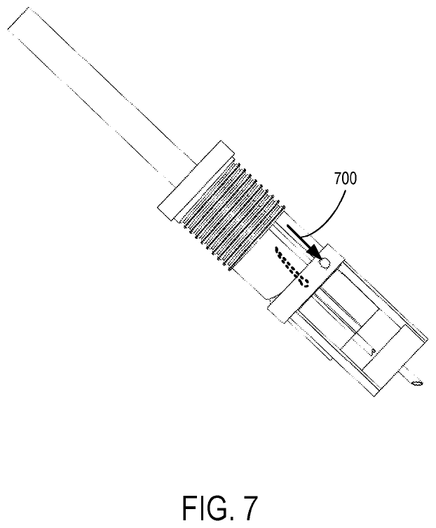

As described above with regard to FIG. 1A, the hair transplant device 100 may include a pin (e.g., pin 140). In operation, the pin's proximal end may connects to a vacuum source (not shown). The hair follicle 610 is extracted into the hair transplant device 100 with suction assistance from the pin, causing the hair follicle 610 to move into a cavity of the extracting unit (e.g., along arrow 620). During performance of operation 504, the hair transplant device 100 may have a configuration as illustrated in FIG. 6. After operation 504, the hair transplant device 100 may be removed from the donor site 600 and operation 506 may be performed to switch the hair transplant device 100 from the extraction state to the implantation state. Operation 506 may include first sliding the implanting unit along the extracting unit with a first coupler of the extracting unit receiving a second coupler of the implanting unit. In the example of FIG. 7, this includes pulling up the implanting unit along the groove of the extracting unit, in the direction of arrow 700. Operation 506 may include next rotating the implanting unit such that the resilient element applies a biasing force to move the second coupler along the first coupler and place the hair transplant system in the implantation state. In the example of FIG. 8, the guide of the implanting unit helps to follow the groove on the extracting unit 110, in the direction of arrow 800. The biasing force from the resilient element (e.g., the spring) will pull the implanting unit along the groove, so that the implanting unit is aligned with the extracting needle. As shown in FIGS. 7 and 8, during operation 506 the hair follicle remains located in the cavity of the extracting unit. This may be facilitated by the continuous application of suction via the pin.

For example, in some instances, during an extraction procedure, the hair transplant device 100 can be used to inject a saline solution under the donor site 600 to make the hair follicles stand up more normal to the donor site 600. In some other instances, the hair transplant device 100 can be used to apply tumescent anesthesia to the donor site 600. The saline solution or tumescent fluid could also be injected using a syringe and a hypodermic needle.

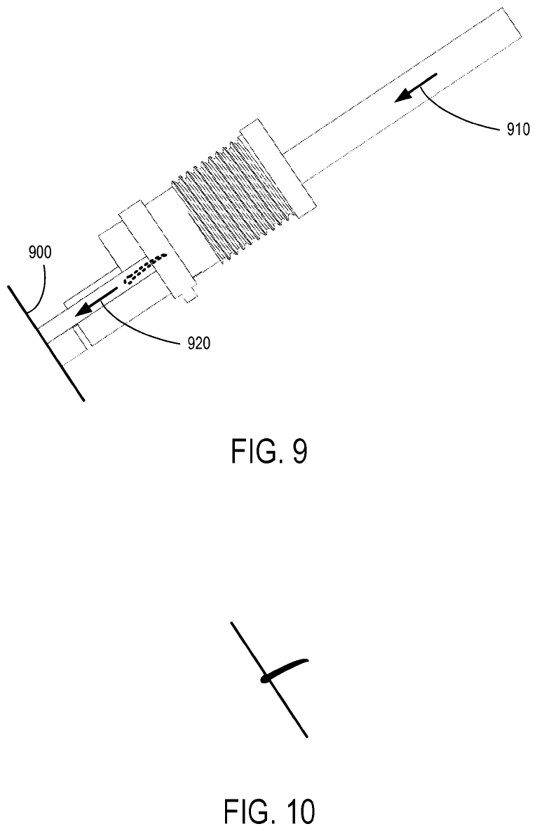

After operation 506, the hair transplant device 100 is in the implantation state, with the implanting needle of the implanting unit seated over the extracting needle. In this configuration, at operation 508 the hair transplant device 100 is pierced at the recipient site for implanting the extracted hair follicle. Next, at operation 510, the hair follicle is implanted at the recipient site 900. As illustrated in FIG. 9, the proximal end of the pin may pushed down (e.g., along the direction of arrow 910) to cause the hair follicle to be pushed out of the cavity (e.g., along the direction of 910), thereby transferring the hair follicle into the recipient site 900 so that the pin stops at the surface of the skin, leaving the hair at a proper depth within the scalp. In some implementations, a small amount (e.g., a few drops) of fluids may be sent through the pin to assist with implanting the hair. After implantation, the hair transplant device 100 is removed from the scalp. As shown in FIG. 10, the hair follicle remains in the scalp at the recipient site.

The method 500 may be repeated any number of times. Between iterations of the method 500, the hair transplant system 100 may be switched from the implantation state back to the extraction state. For example, the implanting unit is turned (e.g., opposite to the arrow 800 of FIG. 8) until the guide is at the highest position of the groove as in FIG. 7 and released. The resilient element will pull down the implanting unit (e.g., opposite to the arrow 700) to the home position/extraction position of operation 502.

As noted above, in some implementations the hair transplant system 100 may include a plurality of extracting needles and implanting needles. In such implementations, the method 500 may proceed as illustrated in FIG. 5. However, in other implementations the hair transplant system 100 may include one extracting needle and a plurality of implanting needles. In such implementations operations 502 and 504 may be performed a plurality of times equal in number to the plurality of implanting needles, each time extracting a new target follicle. After performing operations 502 and 504 the plurality of times, operations 506-510 may be performed in order a single time to implant all hair follicles at once. In still other implementations the hair transplant system 100 may include a plurality of extracting needles and one implanting needles. In such implementations operations 502-506 may be performed once, after which operations 508 and 510 a plurality of times equal in number to the plurality of extracting needles, each time implanting a new follicle.

According to some examples, the hair transplant device 100 may be semi-automated or fully automated. The hair transplant device 100 may include a pin. In one embodiment, a vacuum outlet is in a pin. In alternate embodiments, the pin may be replaced with automated, hydraulic or pneumatic actuation. The hair transplant device 100 can again be configured for use with an automated system. As such, the angle, distribution, and separation between the plurality of hair transplant devices 100 can be controlled by the automated system to effectively control the angle, distribution, and separation between simultaneous extractions, such that every one of the plurality of hair transplant devices 100 extracts an aligned hair follicle. Similarly, the angle, distribution, and separation between the plurality of hair transplant devices 100 can be controlled by the automated system to effectively control the angle, distribution, and separation between simultaneous implantations.

According to some examples, the method 500 of the hair transplant device 100 is a more efficient and cheaper alternative for hair transplants. Extracting hair using the hair transplant device 100 is faster (e.g., twice as fast) as of hair removal Follicular Unit Extraction (“FUE”) and up to five times faster than the overall process of FUE.

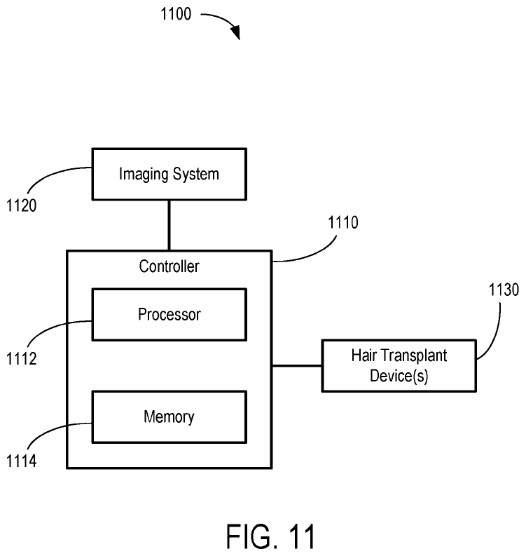

The hair transplant devices described above can be incorporated into an automated hair transplant system. For example, as illustrated in FIG. 11, a hair transplant system 1100 may include a controller 1110 having one or more inputs, processors, memories, and outputs, and may be configured to operate a single hair transplant device 1130 (e.g., the hair transplant device 100 described above) and/or a matrix of hair transplant devices to carry out steps for extracting hair follicles from a donor site and implanting the hair follicles in the recipient site.

The hair transplant system 1100 may include, access, or communicate with one or more user interfaces and/or an imaging system 1120, by way of a wired or wireless connection to the inputs. In various implementations, the hair transplant system 1100 may include any computing device, apparatus or system configured for carrying out instructions and providing input/output capabilities, and may operate as part of, or in collaboration with other computing devices and sensors/detectors (local and remote). In this regard, the hair transplant system 1100 may be a system that is designed to integrate a variety of software and hardware capabilities and functionalities, and/or may be capable of operating autonomously.

The input may include any one or more different input elements, such as a mouse, keyboard, touchpad, touch screen, buttons, and the like, for receiving various selections and operational instructions from a user through touch, movement, speech, etc. The input may also include various drives and receptacles, such as flash-drives, USB drives, CD/DVD drives, and other computer-readable medium receptacles, for receiving various data and information. To this end, input may also include various communication ports and modules, such as Ethernet, Bluetooth, or Wi-Fi, for exchanging data and information with these, and other external computers, systems, devices, machines, mainframes, servers or networks.

In addition to being configured to carry out various steps for operating the hair transplant system, the processor 1112 may be configured to execute instructions, stored in the memory 1114 in a non-transitory computer-readable media. The instructions executable by the processor 1112 may correspond to various instruction for completing a hair transplant procedure (such as those previously described). The memory 1114 may be or include a non-volatile medium, e.g., a magnetic media or hard disk, optical storage, or flash memory; a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; on-chip memory; and/or an installation medium where appropriate, such as software media, e.g., a CD-ROM, or floppy disks, on which programs may be stored and/or data communications may be buffered. Although the non-transitory computer-readable media can be included in the memory 1114, it may be appreciated that instructions executable by the processor 1112 may be additionally or alternatively stored in another data storage location having non-transitory computer-readable media. For example, the hair transplant system 1100 may be configured to implement cloud storage.

As used herein, a “processor” may include one or more individual electronic processors, each of which may include one or more processing cores, and/or one or more programmable hardware elements. The processor may be or include any type of electronic processing device, including but not limited to central processing units (CPUs), graphics processing units (GPUs), application-specific integrated circuits (ASICs), field-programmable gate arrays (FPGAs), microcontrollers, digital signal processors (DSPs), or other devices capable of executing software instructions. When a device is referred to as “including a processor,” one or all of the individual electronic processors may be external to the device (e.g., to implement cloud or distributed computing). In implementations where a device has multiple processors and/or multiple processing cores, individual operations described herein may be performed by any one or more of the microprocessors or processing cores, in series or parallel, in any combination.

In some aspects, the processor 1112 may be configured to receive and process image data from a subject, such as a donor or a recipient, captured by the imaging system 1120 to identify hair follicles and hair follicle orientations within a donor site of the donor and/or to determine implantation locations and necessary implantation angles within a recipient site of the recipient. In some aspects, the processor 1112 may access information and data, including video signals, stored in or emitted by the imaging system 1120. In some aspects, the imaging system 1120 may acquire either a single image or a continuous video signal using, for example, a camera, an infrared scanning system, or any other image capturing or video recording device that can be used to periodically image and/or scan and/or continuously record the subject.

In some instances, the imaging system 1120 may be utilized to align the coring needles of the hair transplant devices 1130 along a hair shaft or a plurality of hair shafts. In some non-limiting examples, the imaging system 1120 can include a camera such as a standard complementary metal-oxide-semiconductor (CMOS) camera, a charge-coupled device (CCD) camera, or an optical coherence tomography (OCT) imaging device. The OCT imaging device may allow for more precise alignment of the coring needles with reference to the hair shafts due to the capability of OCT imaging to see vertically into the tissue. Once the skin cores have been extracted, the hair transplant device 1130, under control of the automated hair transplant system 1100, may position itself over the recipient site for implantation of the hairs. A computer image may similarly be obtained of the recipient site that may show a natural hair line for the patient and direct where the hairs should be implanted. The ability of the needles to move independently may allow for better shaping and following of a natural hair line. In some instances, the patient may be positioned in a support holder or laying down to limit movement during this process.

The output of the hair transplant system 1100 is configured to effectuate the operation of the hair transplant devices 1130. As such, the output may include various robotic devices capable of manipulating and operating the hair transplant devices 1130 and the interface features thereof, to effectuate extraction of hair follicles from a donor site, creation of openings within the recipient, and implantation of the hair follicles within the openings of the recipient, as described above. As such, a user, such as a doctor or other hair transplant procedure personnel, can interact with a user interface of the hair transplant system 1100 to command the automated hair transplant system 1100 to effectuate a hair transplant procedure on a subject in accordance with any of the devices and methods described herein.

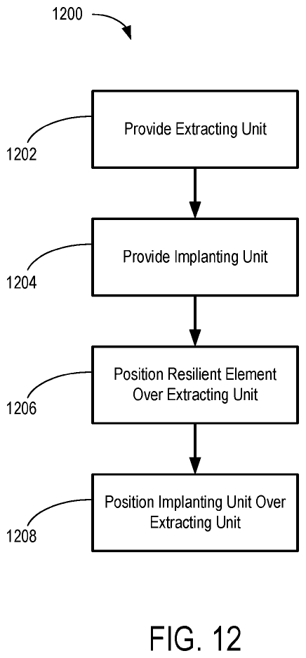

FIG. 12 depicts a method 1200 of manufacturing and assembly of a hair transplant device, such as the hair transplant device 100. The method 1200 includes a first operation 1202 of providing an extracting unit. The extracting unit may be the extracting unit 110 illustrated in FIG. 1A, and may include a first housing (e.g., 112, FIG. 1B) extending from a first proximal end portion to a first distal end portion, wherein the first distal end portion includes an end notch (e.g., 114, FIG. 1B) defining a seat portion and a step portion; an extracting needle (e.g., 116, FIG. 1B) located at the first distal end portion; and a first coupler (e.g., 118, FIG. 1B).

The method 1200 includes a second operation 1204 of providing an implanting unit, which may be the implanting unit 120 illustrated in FIG. 1A. The implanting unit may thus include a second housing (e.g., 122, FIG. 1B) extending from a second proximal end portion to a second distal end portion; an implanting needle (e.g., 124, FIG. 1B) located at the second distal end portion; and a second coupler (e.g., 126, FIG. 1B). Operations 1202 and 1204 may be performed serially in either order, or may be performed in parallel. After operations 1202 and 1204, at operation 1206 a resilient element (e.g., a spring) is inserted over the housing of the extracting unit. Then, at operation 1208, the implanting unit may be positioned over the housing of the extracting unit. In performing operation 1208, the implanting unit may be positioned such that the second coupler is received by the first coupler and such that the resilient element is disposed between the extracting unit and the implanting unit, thereby to provide a biasing force to cause the hair transplant system to move between an extraction state (e.g., of FIG. 4) and an implantation state (e.g., of FIG. 1A).

One or more components of the system may be configured for connection with an automated system, such as, for example a computer-aided manufacturing (CAM) system, for automated use of the hair transplant device 100. The system may additionally be configured for connection with several other similar hair transplant devices, such that an array of hair transplant devices similar to the hair transplant device 100 is provided to allow for automated extraction and/or implantation of multiple hair follicles in series or simultaneously. In some instances, the system may additionally or alternatively be configured for manual manipulation (e.g., can include a handle).

As such, the devices, systems, and methods described herein allow for a user to extract at least one hair follicle from a donor site, and implant the at least one hair follicle using a single device without the need for any physical manipulation of the at least one hair follicle. Accordingly, these devices, systems, and methods allow for more efficient, reliable, and predictable hair transplant procedures than compared to traditional devices, systems, and methods.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

What is claimed is:

1. A hair transplant system, comprising:
an extracting unit including:
a first housing extending from a first proximal end portion to a first distal end portion, wherein the first distal end portion includes an end notch defining a seat portion and a step portion,
an extracting needle located at the first distal end portion, and
a first coupler;
an implanting unit including:
a second housing extending from a second proximal end portion to a second distal end portion,
an implanting needle located at the second distal end portion, and
a second coupler configured to be received by the first coupler; and
a resilient element disposed between the extracting unit and the implanting unit, the resilient element configured to provide a biasing force to cause the hair transplant system to move between an extraction state and an implantation state, wherein
in the extraction state, the second proximal end portion is positioned within the seat portion of the first distal end portion, and
in the implantation state, the second proximal end portion is positioned on the step portion of the first distal end portion.

2. The hair transplant system of claim 1, further comprising a pin, wherein:
in the extraction state, actuation of the pin is configured to cause the hair transplant system to extract a hair from a donor site using the extracting needle, and
in the implantation state, actuation of the pin is configured to cause the hair transplant system to implant a hair at a recipient site using the implantation needle.

3. The hair transplant system of claim 2, wherein the pin includes a port configured for connection to a vacuum source, and a central lumen in fluid communication with a cavity of the extracting unit configured to receive the hair from the donor site.

4. The hair transplant system of claim 1, wherein the first coupler is a groove formed in the first housing, and the second coupler is a guide protrusion configured to be received in the groove.

5. The hair transplant system of claim 1, wherein the resilient element is a spring.

6. The hair transplant system of claim 1, wherein the extracting needle is a coring needle.

7. The hair transplant system of claim 1, wherein the implanting needle is a splitting needle.

8. The hair transplant system of claim 1, wherein, in the implantation state, the implanting needle is seated over the extracting needle.

9. The hair transplant system of claim 1, wherein at least one of the implanting needle or the extracting needle is disposable.

10. The hair transplant system of claim 1, wherein the implanting needle is one of a plurality of implanting needles located at the second distal end portion.

11. A hair transplant method, comprising:
providing a hair transplant device at donor site, wherein the hair transplant system comprises an extracting unit including an extracting needle, an implanting unit including an implanting needle, and a resilient element disposed between the extracting unit and the implanting unit and configured to cause the hair transplant device to move between an extraction state and an implantation state, wherein the hair transplant device is provided in the extraction state;
piercing a target hair follicle at the donor site with the extracting needle;
extracting the target hair follicle into the hair transplant device; and
switching the hair transplant device from the extraction state to the implantation state;
piercing a recipient site with the implantation needle; and
implanting the target hair follicle at the recipient site.

12. The method of claim 11, wherein extracting the target hair follicle includes applying a suction via a pin of the hair transplant device, the pin including a lumen in fluid communication with a cavity of the extracting unit.

13. The method of claim 12, wherein implanting the target hair follicle includes actuating the pin to cause the target hair follicle to move from the cavity to the recipient site.

14. The method of claim 11, wherein the implanting needle is one of a plurality of implanting needles, the method comprising:
repeating the operations of piercing at the donor site and extracting a plurality of times for a plurality of target hair follicles prior to the operation of switching; and
implanting all of the plurality of target hair follicles simultaneously.

15. The method of claim 11, wherein the operation of switching comprises:
sliding the implanting unit along the extracting unit with a first coupler of the extracting unit receiving a second coupler of the implanting unit; and
rotating the implanting unit such that the resilient element applies a biasing force to move the second coupler along the first coupler and place the hair transplant system in the implantation state.

16. The method of claim 11, wherein:
a distal end portion of the extracting unit includes an end notch defining a seat portion and a step portion;
in the extraction state, a proximal end portion of the implanting unit is positioned within the seat portion; and
in the implantation state, the proximal end portion is positioned on the step portion.

17. The method of claim 11, further comprising:
after the operation of implanting, repeating the operations of piercing at the donor site, extracting, switching, piercing at the recipient site, and implanting.

18. The method of claim 11, wherein, in the implanting state, the implanting needle is exposed and, in the extraction state, the implanting needle is not exposed.

19. A method of manufacturing a hair transplant system, comprising:
providing an extracting unit, wherein the extracting unit includes:
a first housing extending from a first proximal end portion to a first distal end portion, wherein the first distal end portion includes an end notch defining a seat portion and a step portion,
an extracting needle located at the first distal end portion, and
a first coupler;
providing an implanting unit, wherein the implanting unit includes:
a second housing extending from a second proximal end portion to a second distal end portion,
an implanting needle located at the second distal end portion, and
a second coupler;
positioning a resilient element over the first housing; and
positioning the implanting unit over the first housing such that the second coupler is received by the first coupler and such that the resilient element is disposed between the extracting unit and the implanting unit, thereby to provide a biasing force to cause the hair transplant system to move between an extraction state and an implantation state, in which:
in the extraction state, the second proximal end portion is positioned within the seat portion of the first proximal end portion, and
in the implantation state, the second proximal end portion is positioned on the step portion of the first proximal end portion.

20. A hair transplant method, comprising:
extracting a target hair follicle from a donor site into a hair transplant device with an extracting needle of a hair transplant device;
positioning an implanting needle of the hair transplant device over the extracting needle without manipulating the target hair follicle; and
implanting the target hair follicle at a recipient site with the implanting needle.
